(19) United States
(12) Patent Application Publication
(10) Pub. No.: US 2025/0188216 A1
(43) Pub. Date: Jun. 12, 2025

(54) FLUORINATED THERMOPLASTIC POLYURETHANE

(71) Applicants: ABIOMED, Inc., Danvers, MA (US); Abiomed Europe GmbH, Aachen (DE)

(72) Inventors: Smriti Singh, Aachen (DE); Claudia Mourran, Aachen (DE)

(73) Assignees: ABIOMED, Inc., Danvers, MA (US); Abiomed Europe GmbH, Aachen (DE)

(21) Appl. No.: 18/976,503

(22) Filed: Dec. 11, 2024

Related U.S. Application Data
(60) Provisional application No. 63/608,954, filed on Dec. 12, 2023.

(51) Int. Cl. C08G 18/77; C08G 18/32

(52) U.S. Cl. CPC C08G 18/773; C08G 18/3215

(57) ABSTRACT

A fluorinated thermoplastic polyurethane (FTPU) for use with medical devices is disclosed. The FTPU may include a soft segment (which may include a polyol), a hard segment (which may include a diisocyanate), and a chain extender. At least one fluorinated portion may be present in the FTPU. The fluorinated portion(s) may include a fluorinated polyol, a fluorinated diisocyanate, a fluorinated chain extender, or a combination thereof. The fluorinated portion(s) may be present in a total amount of 5% to 50% by weight of the FTPU. The FTPU may be used to reduce the coefficient of friction or reduce thrombogenicity of a medical device. A composition of matter may be provided, including a FTPU which may be a reaction product of a polyol, a diisocyanate, a chain extender, and optionally, a catalyst, where at least one of the polyol, diisocyanate, and/or chain extender may be fluorinated.

### CROSS-REFERENCE TO RELATED APPLICATIONS

The present application claims priority to U.S. Provisional Patent Application No. 63/608,954, filed Dec. 12, 2023, the contents of which are incorporated by reference herein in its entirety.

### TECHNICAL FIELD

The present application is drawn to thermoplastic polyurethanes, and specifically to fluorinated thermoplastic polyurethanes useful for medical devices.

### BACKGROUND

Medical devices, including blood pumps and sheaths of various types, utilize a thermoplastic polyurethane (TPU) in one or more parts or components of the device.

### BRIEF SUMMARY

In various aspects, a fluorinated thermoplastic polyurethane (FTPU) may be provided. The FTPU may include a soft segment, a hard segment, and a chain extender. The soft segment may include a polyol. The hard segment may include a diisocyanate. At least one fluorinated portion may be present in the fluorinated thermoplastic polyurethane. The at least one fluorinated portion may include a fluorinated polyol, a fluorinated diisocyanate, a fluorinated chain extender, or a combination thereof. The at least one fluorinated portion may be present in a total amount of 5% to 50% by weight of the fluorinated thermoplastic polyurethane.

At least one fluorinated chain extender may be present in a side chain of the fluorinated thermoplastic polyurethane. At least one fluorinated chain extender may be present in a main chain of the fluorinated thermoplastic polyurethane. The at least one fluorinated chain extender may have an aliphatic, aromatic, or alkoxyl backbone. The at least one fluorinated chain extender may include a diol, a diamine, or both.

The chain extender may include: 1H, 1H, 8H, 8H-perfluoro-3,6-dioxaoctane-1,8 diol; 1H,1H, 11H,11H-perfluoro, 3,6,9-trioxaundecane-1,11-diol; 1H, 1H,2H,3H,3H-perfluorononane-1,2-diol; 1H, 1H, 10H, 10H-perfluoro-1,10 decanediol; 1H, 1H, 6H, 6H-perfluoro-1,6 hexandiol; 1H, 1H, 7H, 7H-perfluoro-1,7 heptadiol; 1H, 1H, 8H, 8H-perfluoro-1,8 octadiol; 1H, 1H, 9H, 9H-perfluoro-1,9 nonodiol; 1H, 1H, 12H, 12H-perfluoro-1,12 dodecanediol; 1H, 1H, 16H, 16H-perfluoro-1,16 hexadecanediol; 2,2,3,3-tetrafluoro-1,4 butanediol; 1,3-Butanediol, 4,4,4-trifluoro-3-(trifluoromethyl); 2,2-Bis(3-amino-4-hydroxyphenyl) hexafluoropropane; 4,4′-(Hexafluoroisopropylidene) dianiline; 4,4′-(Hexafluoroisopropylidene) diphenol; 4,4′-(Hexafluoroisopropylidene) bis (p-phenyleneoxy) dianiline; 4,4′-Bis(4-amino-2-trifluoromethylphenoxy) biphenyl; or a combination thereof.

The chain extender may include a fluorinated parylene-like macromonomer or oligomer. The fluorinated parylene-like macromonomer or oligomer may include 1,4-benzendimethanol; 2,3,5,6-Tetrafluoro-1,4-benzenedimethanol; or a combination thereof.

The diisocyanate may be aromatic, aliphatic, cycloaliphatic or polycyclic in structure. At least one fluorinated diisocyanate may be present. The at least one fluorinated diisocyanate may be aliphatic or aromatic. The at least one fluorinated diisocyanate may include: 2,2-Bis(4-isocyanatophenyl) hexafluoropropane; tetrafluorophenylene-p-diisocyanate; or a combination thereof.

The diisocyanate may be toluene diisocyanate (TDI), methylene diphenyl diisocyanate (MDI), 4,4′-dicyclohexylmethane diisocyanate (H12MDI), xylene diisocyanate (XDI), tetramethylxylylene diisocyanate (TMXDI), hydrogenated xylene diisocyanate (HXDI), naphthalene 1,5-diisocyanate (NDI), p-phenylene diisocyanate (PPDI), 3,3′-dimethyldiphenyl-4, 4′-diisocyanate (DDDI), hexamethylene diisocyanate (HDI), 2,2,4-trimethylhexamethylene diisocyanate (TMDI), isophorone diisocyanate (IPDI), norbornane diisocyanate (NDI), 4,4′-dibenzyl diisocyanate (DBDI), or a combination thereof.

At least one fluorinated polyol may be present. The at least one fluorinated polyol may include a fluorine-containing copolymer grafted to the polyol. The fluorine-containing copolymer may be a random copolymer. The fluorine-containing copolymer may be a block copolymer. The fluorine-containing copolymer may have perfluoroalkyl side chains. The fluorine-containing copolymer may have at least one side group that may include —CH₂(CF₂)ₙCF₃, where n may be no more than 6. In some embodiments, n may be 0, 2, 3, or 6.

The at least one fluorinated polyol may be, e.g., poly(glycidol)-co-(polypropylene) grafted with 4-Fluor-3-(trifluormethyl)phenylisocyanate. A ratio of glycidol to 4-fluor-3-(trifluormethyl)phenylisocyanate may be no more than 20%.

The polyol may be a polyfunctional polyether, a polyester polyol (PEPO), an acrylic polyol (ACPO), a polycarbonate polyol, castor oil, or a combination thereof. The polyol may include ethylene glycol; polyethylene glycol; polypropylene glycol; 1,4-butane diol (BDO); 1,6-hexane diol; or a combination thereof.

In various aspects, a method for producing a medical device may be provided. The method may include providing an embodiment of an FTPU as disclosed herein, and forming a portion of the medical device utilizing the fluorinated thermoplastic polyurethane.

In various aspects, a medical device may be provided, the medical device including an embodiment of an FTPU as disclosed herein.

The medical device may be, e.g., an introducer sheath, a repositioning sheath, or a blood pump. The medical device may include a component utilizing the FTPU. The component may be, e.g., a cannula, catheter, sheath body, inflow cage, outflow tube, distal extension, or dilator tip or a combination of inflow cannula and outflow cannula

In various aspect, embodiments of the FTPU may be used to reduce the coefficient of friction or reduce thrombogenicity of a medical device. For example, if an internal or external surface of the medical device comprises the FTPU, that surface may have a reduced coefficient of friction or a reduced thrombogenicity.

In various aspects, a composition of matter may be provided. The composition of matter may include an FTPU. The FTPU may be a reaction product of a polyol, a diisocyanate, a chain extender, and optionally, a catalyst. At least one of the polyol, diisocyanate, and/or chain extender may be fluorinated.

### BRIEF DESCRIPTION OF FIGURES

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is a simplified schematic of a FTPU.

FIGS. 2A-2B are illustrations of different blood pumps.

FIG. 3 is an illustration of an introducer sheath assembly.

FIG. 4 is an illustration of a cross-section of a sheath and hub with a sideport.

FIG. 5 is an illustration of a dilator assembly.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

As is known in the art, thermoplastic polyurethanes (TPUs) are generally synthesized with a soft segment diol, a hard segment diisocyanate, and a chain extender. The incompatibility between soft and hard segments results in microphase separation, the structure of which dominates the mechanical properties and phase morphology of the TPU. However, many properties of TPU are not ideal for medical devices.

For example, it may be desirable to provide a TPU with properties such as a relatively low interfacial free energy as well as improved water resistance when used for application in medical devices. In some embodiments, it may be desirable to have lower coefficient of friction and/or reduced thrombogenicity.

To accomplish this, one approach may be for fluorine-containing portions (such as hard and/or soft segments) to be introduced into TPUs to achieve segmented fluorinated TPUs (FTPUs) by (for example) introducing fluorinated blocks into the molecular chains of TPU. The process can be applied to FTPUs synthesized in one-step bulk polymerization as well as for FTPUs produced by 2 component casting process. An FTPU can be obtained with outstanding properties, such as high strength, high toughness, high damping properties, improved solvent and chemical resistance, a lower surface tension, and low coefficient of friction.

The well separated phase structure and larger changes in the FTPU's surface energy (compared to a non-fluorinated comparable TPU) because of fluorinated chains segregating towards the surface will have a significant effect on protein adsorption and subsequent coagulation events in blood/material interfacing systems.

Fluorine could be incorporated into a TPU in one or more ways, some examples of which are as follows. Short or long segmented fluorine-containing chain extenders can be incorporated into the main and side chains of TPU. Fluorine-containing diisocyanate could be introduced in the polyurethane manufacturing process. Fluorinated side groups can be introduced in polyols: fluorine-containing copolymers (random or block) with long perfluoroalkyl side chains can be grafted to the polyol. ‘parylene-like’ macromonomers or oligomers can be introduced as chain extenders into the main or side chains of the TPU.

As used herein, the term “aliphatic” generally refers to material containing a carbon-carbon double bond including linear (or open chain) aliphatic carbon-carbon double bonds and/or cycloaliphatic carbon-carbon double bonds, but excludes aromatic carbon-carbon double bonds of aromatic rings. As used herein, the term “cycloaliphatic” refers to, e.g., a cyclic hydrocarbon that is completely saturated or that contains one or more units of unsaturation, but which is not aromatic.

As used herein, the term “alkoxyl” generally refers to a stable straight or branched chain, or cyclic hydrocarbon radical, or combinations thereof, containing a number of carbon atoms and at least one (in one embodiment, one to three) O atoms. Examples of alkoxyl include, but are not limited to, —O—CH₃, —O—CF₃, —O—CH₂—CH₃, —OCH₂—CH₂—CH₃, —O—CH—(CH₃)₂, and —O—CH₂—CH₂—O—CH₃. In one embodiment, the alkoxyl may be optionally substituted.

As used herein, the term “aromatic” generally refers to any material containing aryl and/or heteroaryl groups optionally substituted with functional groups.

As used herein, the term “parylene-like macromonomer or oligomer” refers to a macromonomer or oligomer having a backbone that consists of para-benzenediyl rings (—C₆H₄—) connected by 1,2-ethanediyl bridges (—CH₂—CH₂-). The fluorinated parylene-like macromonomer or oligomer may include 1,4-benzendimethanol; 2,3,5,6-Tetrafluoro-1,4-benzenedimethanol; or a combination thereof.

As used herein, the term “polymer backbone” or “main chain” refers to the covalently bonded chain of repeating monomer units that form the polymer. The polymer backbone may be covalently attached to terminal functional groups or pendant functionalized side chains spaced along the polymer backbone.

As used herein, the term “polymer side chain” or “side chain” refers to the portion of the monomer that forms an extension off the polymer backbone.

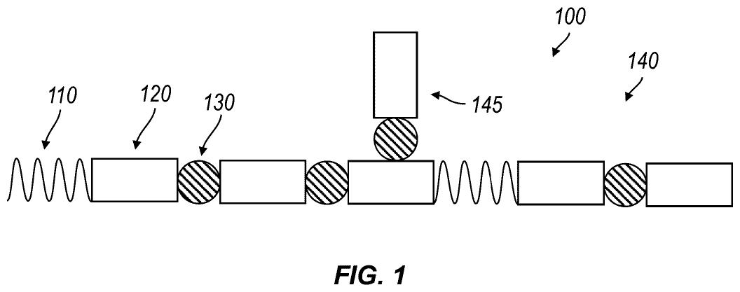

In various aspects, a fluorinated thermoplastic polyurethane (FTPU) may be provided. Referring to FIG. 1, the FTPU (100) may include a soft segment (110), a hard segment (120), and a chain extender (130). As used herein, the term “hard segment” refers to a segment formed of a material harder than a material constituting the soft segment in the FTPU, and the term “soft segment” refers to a segment formed of a material softer than a material constituting the hard segment in the FTPU. The soft segment may include a polyol. The hard segment may include a diisocyanate. At least one fluorinated portion may be present in the fluorinated thermoplastic polyurethane. The at least one fluorinated portion may include a fluorinated polyol, a fluorinated diisocyanate, a fluorinated chain extender, or a combination thereof.

The at least one fluorinated portion may be present in a total amount of at least 1% by weight of the fluorinated thermoplastic polyurethane. The at least one fluorinated portion may be present in a total amount of at least 5% by weight of the fluorinated thermoplastic polyurethane. The at least one fluorinated portion may be present in a total amount of at least 10% by weight of the fluorinated thermoplastic polyurethane. The at least one fluorinated portion may be present in a total amount of at least 15% by weight of the fluorinated thermoplastic polyurethane. The at least one fluorinated portion may be present in a total amount of at least 20% by weight of the fluorinated thermoplastic polyurethane. The at least one fluorinated portion may be present in a total amount of at least 25% by weight of the fluorinated thermoplastic polyurethane. The at least one fluorinated portion may be present in a total amount of at least 30% by weight of the fluorinated thermoplastic polyurethane. The at least one fluorinated portion may be present in a total amount of at least 35% by weight of the fluorinated thermoplastic polyurethane. The at least one fluorinated portion may be present in a total amount of at least 40% by weight of the fluorinated thermoplastic polyurethane. The at least one fluorinated portion may be present in a total amount of at least 45% by weight of the fluorinated thermoplastic polyurethane. The at least one fluorinated portion may be present in a total amount of no more than 60% by weight of the fluorinated thermoplastic polyurethane. The at least one fluorinated portion may be present in a total amount of no more than 50% by weight of the fluorinated thermoplastic polyurethane. The at least one fluorinated portion may be present in a total amount of no more than 45% by weight of the fluorinated thermoplastic polyurethane. The at least one fluorinated portion may be present in a total amount of no more than 40% by weight of the fluorinated thermoplastic polyurethane. The at least one fluorinated portion may be present in a total amount of no more than 35% by weight of the fluorinated thermoplastic polyurethane. The at least one fluorinated portion may be present in a total amount of no more than 30% by weight of the fluorinated thermoplastic polyurethane. The at least one fluorinated portion may be present in a total amount of no more than 25% by weight of the fluorinated thermoplastic polyurethane. The at least one fluorinated portion may be present in a total amount of no more than 20% by weight of the fluorinated thermoplastic polyurethane. The at least one fluorinated portion may be present in a total amount of no more than 15% by weight of the fluorinated thermoplastic polyurethane. The at least one fluorinated portion may be present in a total amount of no more than 10% by weight of the fluorinated thermoplastic polyurethane.

At least one fluorinated chain extender may be present in the main chain (140) and/or a side chain (145) of the fluorinated thermoplastic polyurethane. As used herein, the term “main chain” generally refers to the longest chain of atoms (e.g., carbon atoms) among one or more chains of atoms included in a given polymer. As used herein, the term “side chain” generally refers to any chain of atoms, other than the main chain, included in the polymer.

The at least one fluorinated chain extender may have an aliphatic, aromatic, or alkoxyl backbone. The at least one fluorinated chain extender may include a diol, a diamine, or both.

Non-limiting examples of chain extenders may be composed of one or more of the monomers indicated in Table 1, below.

TABLE 1

| | |
|---|---|
| 1H,1H,8H,8H-perfluoro-3,6- dioxaoctane-1,8 diol | 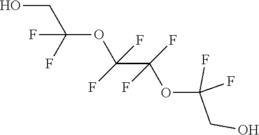 [SMILES: OCC(F)(F)OC(F)(F)C(F)(F)OC(F)(F)CO] |
| 1H,1H,11H,11H-perfluoro,3,6,9- trioxaundecane-1,11-diol | 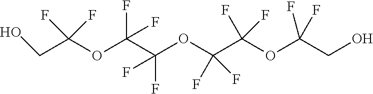 [SMILES: OCC(F)(F)OC(F)(F)C(F)(F)OC(F)(F)C(F)(F)OC(F)(F)CO] |
| 1H,1H,2H,3H,3H-perfluorononane- 1,2-diol | 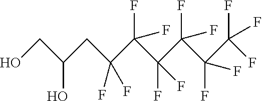 [SMILES: OCC(O)CC(F)(F)C(F)(F)C(F)(F)C(F)(F)C(F)(F)C(F)(F)F] |
| 1H,1H,10H,10H-perfluoro-1,10 decanediol | 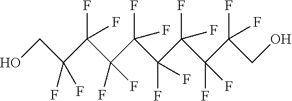 [SMILES: OCC(F)(F)C(F)(F)C(F)(F)C(F)(F)C(F)(F)C(F)(F)C(F)(F)C(F)(F)CO] |
| 1H,1H,6H,6H-perfluoro-1,6 | HO—CH₂—(CF₂)₄—CH₂—OH |
| hexandiol | |
| 1H,1H,7H,7H-perfluoro-1,7 | HO—CH₂—(CF₂)₅—CH₂—OH |
| heptadiol | |
| 1H,1H,8H,8H-perfluoro-1,8 | HO—CH₂—(CF₂)₆—CH₂—OH |
| octadiol | |
| 1H,1H,9H,9H-perfluoro-1,9 | HO—CH₂—(CF₂)₇—CH₂—OH |
| nonodiol | |
| 1H,1H,12H,12H-perfluoro-1,12 | HO—CH₂—(CF₂)₁₀—CH₂—OH |
| dodecanediol | |
| 1H,1H,16H,16H-perfluoro-1,16 | HO—CH₂—(CF₂)₁₄—CH₂—OH |
| hexadecanediol | |
| 2,2,3,3-tetrafluoro-1,4 butanediol | 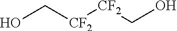 |
| 1,3-Butanediol,4,4,4-trifluoro-3- (trifluoromethyl) | 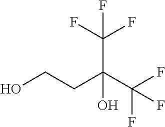 [SMILES: OCCC(O)(C(F)(F)F)C(F)(F)F] |
| 2,2-Bis(3-amino-4- hydroxyphenyl)hexafluoropropane | 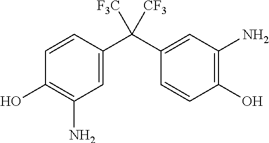 [SMILES: Nc1cc(C(c2ccc(O)c(N)c2)(C(F)(F)F)C(F)(F)F)ccc1O] |
| 4,4′- (Hexafluoroisopropylidene)dianiline | 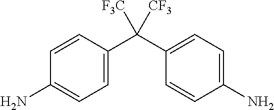 [SMILES: Nc1ccc(C(c2ccc(N)cc2)(C(F)(F)F)C(F)(F)F)cc1] |
| 4,4′- (Hexafluoroisopropylidene)diphenol | 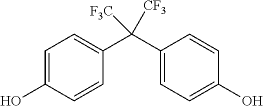 [SMILES: Oc1ccc(C(c2ccc(O)cc2)(C(F)(F)F)C(F)(F)F)cc1] |
| 4,4′- (Hexafluoroisopropylidene)bis(p- phenyleneoxy)dianiline | 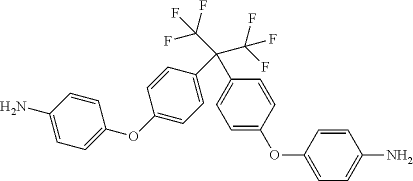 [SMILES: Nc1ccc(Oc2ccc(C(c3ccc(Oc4ccc(N)cc4)cc3)(C(F)(F)F)C(F)(F)F)cc2)cc1] |
| 4,4′-Bis(4-amino-2- trifluoromethylphenoxy)biphenyl | 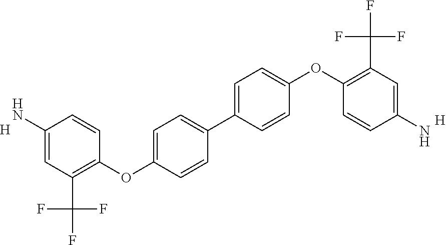 |

The chain extender may include a fluorinated parylene-like macromonomer or oligomer. The parylene-line macromonomer or oligomer may be composed of one or more of the monomers listed in Table 2.

TABLE 2

| | |
|---|---|
| 1,4-benzendimethanol | 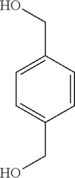 |
| 2,3,5,6-Tetrafluoro-1,4- benzenedimethanol | 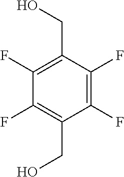 [SMILES: OCc1c(F)c(F)c(CO)c(F)c1F] |

As seen, the parylene-like component may have a composition of HO—R1-CH₂—C₆H₄₋ₓFₓ—CH₂—R2-OH, where R1 and R2 are independently (CH_{2-y}F_{y})ₙOH, where x may be 0-4, y may be 0-2, and n may be 0-6. For example, RI could be CH₂—CF₂—CH₂—OH, R2 could be (CF₂)₂—OH, and the para-benzenediyl ring could include 0-4 fluorine atoms. In some embodiments, n may be no more than 5. In some embodiments, n may be no more than 4. In some embodiments, n may be no more than 3. In some embodiments, n may be no more than 2. In some embodiments, n may be no more than 1. In some embodiments, n may be 0. In some embodiments, x may be at least 1. In some embodiments, x may be 0. In some embodiments, y may be at least 1. In some embodiments, y may be 0.

The diisocyanate may be aromatic, aliphatic, cycloaliphatic or polycyclic in structure. At least one fluorinated diisocyanate may be present. The at least one fluorinated diisocyanate may be aliphatic or aromatic. The at least one fluorinated diisocyanate may be composed of one or more of the monomers listed in Table 3.

TABLE 3

| | |
|---|---|
| 2,2-Bis(4- isocyanatophenyl) hexafluoropropane | 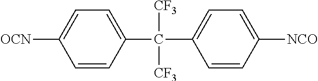 |
| tetrafluorophenylene- p-diisocyanate | 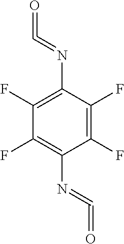 [SMILES: O=C=Nc1c(F)c(F)c(N=C=O)c(F)c1F] |

Non-limiting examples of diisocyanates include toluene diisocyanate (TDI), methylene diphenyl diisocyanate (MDI), 4,4′-dicyclohexylmethane diisocyanate (H12MDI), xylene diisocyanate (XDI), tetramethylxylylene diisocyanate (TMXDI), hydrogenated xylene diisocyanate (HXDI), naphthalene 1,5-diisocyanate (NDI), p-phenylene diisocyanate (PPDI), 3,3′-dimethyldiphenyl-4,4′-diisocyanate (DDDI), hexamethylene diisocyanate (HDI), 2,2,4-trimethylhexamethylene diisocyanate (TMDI), isophorone diisocyanate (IPDI), norbornane diisocyanate (NDI), 4,4′-dibenzyl diisocyanate (DBDI), or a combination thereof.

At least one fluorinated polyol may be present. The at least one fluorinated polyol may include a fluorine-containing copolymer grafted to the polyol. The fluorine-containing copolymer may be a random copolymer. The fluorine-containing copolymer may be a block copolymer. The fluorine-containing copolymer may have perfluoroalkyl side chains. The fluorine-containing copolymer may have at least one side group that may include —CH₂(CF₂)ₙCF₃, where n is no more than 6. In some embodiments, n may be 0, 2, 3, or 6.

The at least one fluorinated polyol may be, e.g., poly(glycidol)-co-(polypropylene) grafted with 4-Fluor-3-(trifluormethyl)phenylisocyanate. In some embodiments, controlling the ratio of glycidol to 4-fluor-3-(trifluormethyl)phenylisocyanate may be useful. The ratio may be at least 5%. The ratio may be at least 10%. The ratio may be at least 15%. The ratio may be at least 20%. The ratio may be at least 25%. The ratio may be no more than 30%. The ratio may be no more than 25%. The ratio may be no more than 20%. The ratio may be no more than 15%. The ratio may be no more than 10%.

The polyol may be a polyfunctional polyether, a polyester polyol (PEPO), an acrylic polyol (ACPO), a polycarbonate polyol, castor oil, or a combination thereof. The polyol may include ethylene glycol; polyethylene glycol; polypropylene glycol; 1,4-butane diol (BDO); 1,6-hexane diol; or a combination thereof.

In various aspects, a composition of matter may be provided. The composition of matter may include an FTPU. The FTPU may be a reaction product of a polyol (as disclosed herein), a diisocyanate (as disclosed herein), a chain extender (as disclosed herein), and optionally, a catalyst. At least one of the polyol, diisocyanate, and/or chain extender may be fluorinated. For example, the FTPU may be a reaction product of 25-95% w/w of a polyol, 5-75% w/w of a diisocyanate, 0.1-5% w/w of a chain extender, and optionally, 0-2% of a catalyst (summing to 100% w/w).

Polymerization catalysts for TPU reactions are well understood in the art. Generally, any conventional catalyst can be utilized to react the diisocyanate with the polyol and/or chain extender. Non-limiting examples of catalysts include organic tin compounds such as dibutyltin diacetate, dibutyltin dilaurate (DBTL), dioctyltin dilaurate (DOTDL), and dibutyltin bis(ethoxybutyl 3-mercaptopropionate); titanic acid; organic titanium compounds such as tetraisopropyl titanate, tetra-n-butyl titanate, polyhydroxytitanium stearate and titanium acetylacetonate; tertiary amines such as triethylene diamine, triethylamine N-methylmorpholine, N,N,N′,N′-tetramethylethylenediamine, N,N,N′,N′-tetramethylhexamethylene diamine, triethylamine, N,N′-dimethylpiperazine, N,N-dimethylaminoethanol, dimethylcyclohexylamine, and diazabicyclo[2.2.2]octane; and appropriate mixtures thereof.

In various aspects, a method for producing a medical device may be provided. The method may include providing an embodiment of an FTPU as disclosed herein, and forming a portion of the medical device utilizing the fluorinated thermoplastic polyurethane.

In various aspects, a medical device may be provided, the medical device including an embodiment of an FTPU as disclosed herein.

The medical device may be, e.g., a blood pump, an introducer sheath, a repositioning sheath, or a blood pump. The medical device may include a component utilizing the FTPU. The component may be, e.g., a cannula, catheter, sheath body, inflow cage, distal extension, or dilator tip.

In various aspect, embodiments of the FTPU may be used to reduce the coefficient of friction or reduce thrombogenicity of a medical device. For example, if an internal or external surface of the medical device comprises the FTPU, that surface may have a reduced coefficient of friction or a reduced thrombogenicity.

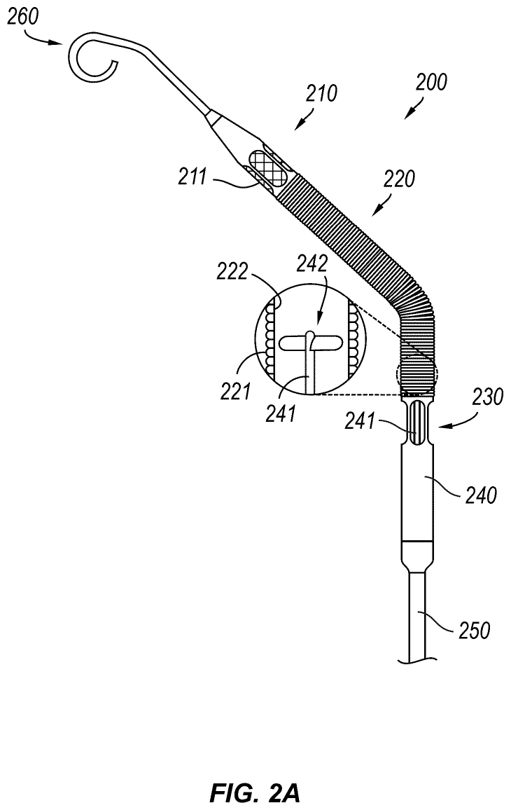

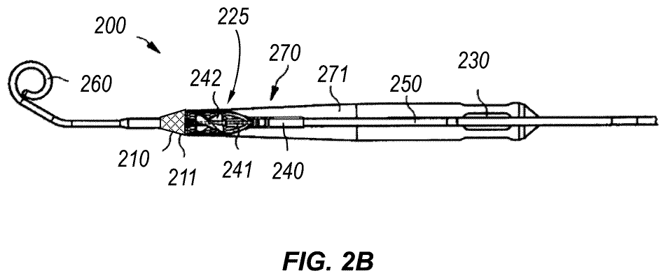

For example, referring to FIG. 2A, a blood pump (200) can be seen, having a cannula (220) operably coupled to a distal end of catheter (250). A flexible atraumatic tip (260) may be coupled to a distal end of the blood pump. The cannula may have an inner surface (222) and an outer surface (221). The outer surface may be exposed to, e.g., blood vessel walls and blood flowing around the blood pump. The inner surface may be expose to, e.g., blood flowing through the cannula. The cannula (220) may, optionally, may include an impeller (242) disposed within the cannula (220). Optionally, the impeller may be within a housing (240). Housing (240) may include, e.g., the impeller and/or an electric motor. The housing (which may sometimes be referred to as a pump housing) may be disposed between the catheter and the cannula.

The motor may be coupled to the impeller via a drive shaft (241), which may be a rigid drive shaft. In some embodiments, when the impeller rotates, blood is configured to flow through an inlet (210), through the cannula, and out an outlet (230). The inlet may have an inflow cage (211) disposed over or at the inlet.

In some embodiment, the distal extension (260), the inflow cage (211), the cannula (220) (including, for example, the inner surface (222) and/or the outer surface (221)), and the catheter (250) may be composed of one or more embodiments of the FTPU. In some embodiments, any FTPU used in the blood pump has a single composition. In some examples, a first part of the blood pump (the cannula) may be configured with an FTPU having a first composition, and a second part of the blood pump (such as the distal extension) may be configured with an FTPU having a second composition different from the first composition.

In FIG. 2B, a different blood pump design can be seen. There, the blood pump (200) is shown as having an expandable pump housing (225) which may include one or more FTPUs. An impeller (242) in the expandable pump housing may be configured to cause blood to flow through the inlet (210), through an intermediate portion of the expandable pump housing, out of the pump housing an into a volume of space (271) defined by an outflow tube (270). The outflow tube may include one or more FTPUs. The blood then exits at the end of the outflow tube, through an outlet (230).

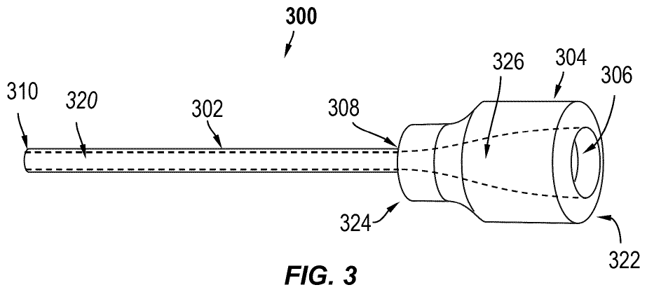

In FIG. 3, an expandable introducer sheath assembly can be seen. Introducer sheath (300) may have a sheath body (302) defined by a proximal end (308), a distal end (310), and a lumen (320) extending through the sheath body between the proximal and distal end. On the proximal end (308), the expandable sheath body (302) may be attached to a hub (304). The hub (304) may include a proximal end (322) and a distal end (324), with a lumen (326) extending between the proximal end and the distal end. On its distal end, the hub (304) may be attached to the expandable sheath body (302). On the proximal side of the hub (304) there may be a hemostasis valve (306) within the sheath hub (304). The hemostasis valve (306) may allow for insertion of components through the hub and sheath but prevents flow of fluid (i.e. blood) from the distal end of the expandable sheath body (302) to the outside of the expandable sheath body (302) and hub (304). The hub (304) may include a side-arm (not shown in FIG. 3) that allows for aspiration of fluid and flushing of the sheath. In some embodiments, the sheath both and/or portion of the hub may include one or more embodiments of the FTPU.

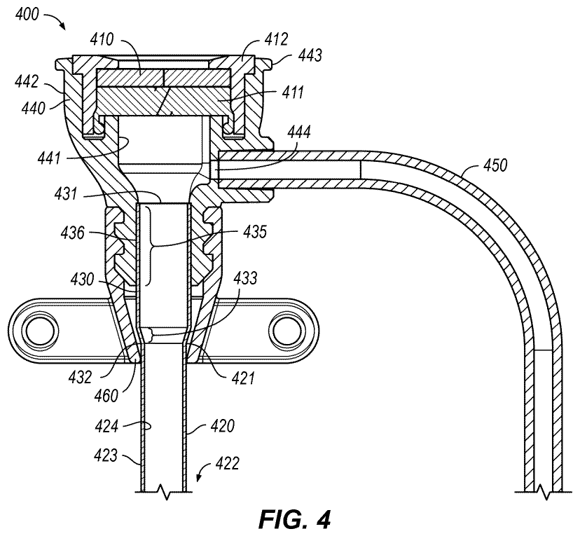

Referring to FIG. 4, a device (400) can be seen. The sheath may be used for, e.g., insertion into a vasculature of a patient. The device may include a primary sheath body (420) operably coupled to a distal end of an intermediate sheath body (430), and a hub (440) coupled to a proximal portion of the intermediate tubular sheath body (430). The hub (440) may be connected to a sideport (450). A rotatable portion (460) or member may be operably coupled to a distal end of the hub.

The sheath both (420) may extend from a proximal end (421) to a distal end (422), and may include one or more layers, such as an outer jacket (423) and an inner frame layer (424). The intermediate sheath body (430) may extend from a proximal end 431 to a distal end (432). In some embodiments, the intermediate tubular sheath body (430) may have a proximal portion (435) with a constant inner diameter and a distal portion (433) that necks down linearly from the inner diameter of the proximal portion to a smaller inner diameter at the distal end (432).

The hub (440) may be connected to a proximal portion (436) of the intermediate sheath body. An inner surface (441) of the hub (440) may be connected to a portion of the outer surface (436) of the intermediate tubular sheath body (430) and not connected with the primary tubular sheath body (420).

The sideport (450) may extend from the hub (440). A first end of the sideport may be connected an entrance defined by an exterior surface (442) of the hub (440) that extends to the interior surface (441) of the hub. The device may also comprise a suture portion (460) connected to a distal portion of the hub (440).

The device may include a hemostatic valve assembly. The hemostatic valve assembly may include, e.g., a foam member (410) configured to secure an elastomeric member (411) at least partially within a housing (412). The housing may be disposed with hub (440).

In some embodiments, the primary sheath, the intermediate sheath, the hub (e.g., at the inner surface), and/or the side port may be composed of one or more embodiments of an FTPU.

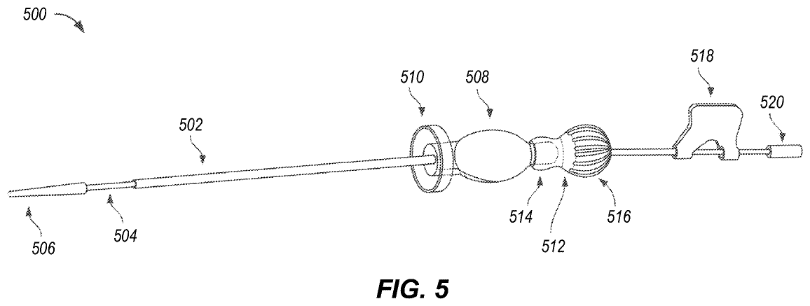

FIG. 5 shows a dilator assembly (500) with an outer dilator (502), inner dilator (504), hub attachment (508), and tip interlock (518). The distal end of the inner dilator (504) consists of a tube with a distal end, a proximal end, and a lumen defined through. It is bonded to a distal tip (e.g., dilator tip (506)), where the proximal end of the inner dilator (504) may be bonded to, e.g., a luer hub 520. The outer dilator (502) is axially aligned with the inner dilator (504) and has a length less than the inner dilator (504) such that the inner dilator (504) is exposed on either end, and slides within the outer dilator (502). The hub attachment (508) consists of a hub attachment cap (510) that contains features to attach to the hub (504) of a sheath on the distal end and may be bonded to, e.g., a luer assembly (514) containing a compressible elastomer (512) and a compression nut (516). When the compression nut (516) is loose, the compressible elastomer (512) is in a first state of minimum compression, allowing the hub attachment (508) to slide along the outer dilator (502). When the compression nut (516) is tightened, the compressible elastomer (512) is in a second state of maximum compression, preventing the hub attachment (508) from sliding on the outer dilator (502) and locking the hub attachment (508) in place with respect to the outer dilator (502).

In some embodiments, the distal tip, inner dilator, and/or outer dilator may be composed of one or more FTPUs.

Various modifications may be made to the systems, methods, apparatus, mechanisms, techniques, and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof. As such, the appropriate scope of the invention is to be determined according to the claims.

1. A fluorinated thermoplastic polyurethane, comprising:
a soft segment comprising a polyol;
a hard segment comprising a diisocyanate; and
a chain extender;
wherein at least one fluorinated portion is present in the fluorinated thermoplastic polyurethane, the at least one fluorinated portion includes a fluorinated polyol, a fluorinated diisocyanate, a fluorinated chain extender, or a combination thereof.

2. The fluorinated thermoplastic polyurethane of claim 1, wherein the at least one fluorinated portion is present in a total amount of 5% to 50% by weight of the fluorinated thermoplastic polyurethane.

3. The fluorinated thermoplastic polyurethane of claim 1, wherein at least one fluorinated chain extender is present in a side chain of the fluorinated thermoplastic polyurethane.

4. The fluorinated thermoplastic polyurethane of claim 3, wherein the at least one fluorinated chain extender has an aliphatic, aromatic, or alkoxyl backbone.

5. The fluorinated thermoplastic polyurethane of claim 1, wherein at least one fluorinated chain extender is present in a main chain of the fluorinated thermoplastic polyurethane.

6. The fluorinated thermoplastic polyurethane of claim 5, wherein the at least one fluorinated chain extender has an aliphatic, aromatic, or alkoxyl backbone.

7. The fluorinated thermoplastic polyurethane of claim 1, wherein the at least one fluorinated chain extender includes a diol, a diamine, or both.

8. The fluorinated thermoplastic polyurethane of claim 1, wherein the chain extender comprises: 1H, 1H, 8H, 8H-perfluoro-3,6-dioxaoctane-1,8 diol; 1H,1H, 11H,11H-perfluoro, 3,6,9-trioxaundecane-1,11-diol; 1H, 1H,2H,3H,3H-perfluorononane-1,2-diol; 1H, 1H, 10H, 10H-perfluoro-1,10 decanediol; 1H, 1H, 6H, 6H-perfluoro-1,6 hexandiol; 1H, 1H, 7H, 7H-perfluoro-1,7 heptadiol; 1H, 1H, 8H, 8H-perfluoro-1,8 octadiol; 1H, 1H, 9H, 9H-perfluoro-1,9 nonodiol; 1H, 1H, 12H, 12H-perfluoro-1,12 dodecanediol; 1H, 1H, 16H, 16H-perfluoro-1,16 hexadecanediol; 2,2,3,3-tetrafluoro-1,4 butanediol; 1,3-Butanediol, 4,4,4-trifluoro-3-(trifluoromethyl); 2,2-Bis(3-amino-4-hydroxyphenyl) hexafluoropropane; 4,4′-(Hexafluoroisopropylidene) dianiline; 4,4′-(Hexafluoroisopropylidene) diphenol; 4,4′-(Hexafluoroisopropylidene) bis (p-phenyleneoxy) dianiline; 4,4′-Bis(4-amino-2-trifluoromethylphenoxy) biphenyl; or a combination thereof.

9. The fluorinated thermoplastic polyurethane of claim 1, wherein the chain extender comprises a fluorinated parylene-like macromonomer or oligomer.

10. The fluorinated thermoplastic polyurethane of claim 9, wherein the fluorinated parylene-like macromonomer or oligomer comprises 1,4-benzendimethanol; 2,3,5,6-Tetrafluoro-1,4-benzenedimethanol; or a combination thereof.

11. The fluorinated thermoplastic polyurethane of claim 1, wherein at least one fluorinated diisocyanate is present.

12. (canceled)

13. (canceled)

14. The fluorinated thermoplastic polyurethane of claim 1, wherein the diisocyanate is aromatic, aliphatic, cycloaliphatic or polycyclic in structure.

15. The fluorinated thermoplastic polyurethane of claim 1, wherein the diisocyanate is toluene diisocyanate (TDI), methylene diphenyl diisocyanate (MDI), 4,4′-dicyclohexylmethane diisocyanate (H12MDI), xylene diisocyanate (XDI), tetramethylxylylene diisocyanate (TMXDI), hydrogenated xylene diisocyanate (HXDI), naphthalene 1,5-diisocyanate (NDI), p-phenylene diisocyanate (PPDI), 3,3′-dimethyldiphenyl-4,4′-diisocyanate (DDDI), hexamethylene diisocyanate (HDI), 2,2,4-trimethylhexamethylene diisocyanate (TMDI), isophorone diisocyanate (IPDI), norbornane diisocyanate (NDI), 4,4′-dibenzyl diisocyanate (DBDI), or a combination thereof.

16. The fluorinated thermoplastic polyurethane of claim 1, wherein at least one fluorinated polyol is present.

17-24. (canceled)

25. The fluorinated thermoplastic polyurethane of claim 1, wherein the polyol is a polyfunctional polyether, a polyester polyol (PEPO), an acrylic polyol (ACPO), a polycarbonate polyol, castor oil, or a combination thereof.

26. The fluorinated thermoplastic polyurethane of claim 1, wherein the polyol comprises ethylene glycol; polyethylene glycol; polypropylene glycol; 1,4-butane diol (BDO); 1,6-hexane diol; or a combination thereof.

27. A method for producing a medical device, comprising:
providing a fluorinated thermoplastic polyurethane of claim 1; and
forming a portion of the medical device utilizing the fluorinated thermoplastic polyurethane.

28. A medical device comprising a fluorinated thermoplastic polyurethane of claim 1.

29-30. (canceled)

31. The use of a fluorinated thermoplastic polyurethane of claim 1 to reduce the coefficient of friction or reduce thrombogenicity of a medical device.

32. A composition of matter, comprising:
a fluorinated thermoplastic polyurethane polymer which is a reaction product of:
a polyol;
a diisocyanate;
a chain extender; and
optionally, a catalyst;
wherein at least one of the polyol, diisocyanate, and/or chain extender is fluorinated.
